(19) United States
(12) Patent Application Publication
(10) Pub. No.: US 2011/0160728 A1
(43) Pub. Date: Jun. 30, 2011

(54) Intramedullary Compression Nail and Related Method for Jones Fractures

(75) Inventors: Neal Blitz, New York, NY (US); Michael Thomas, Van Alstyne, TX (US); David Crook, Mineola, TX (US); Paul Vasta, McKinney, TX (US)

(73) Assignee: AMEI TECHNOLOGIES, INC., Wilmington, DE (US)

(21) Appl. No.: 12/651,371

(22) Filed: Dec. 31, 2009

(51) Int. Cl. A61B 17/58

(52) U.S. Cl. 606/64

(57) ABSTRACT

An intramedullary fixation device and related method for treatment of a Jones fracture. An exemplary device may comprise a rod having a distal section and a threaded proximal section, and means for securing the distal section within a first bone portion on a distal side of a fracture site. The device may also comprise a compression collar threadably received over the proximal section for providing compression to the fracture site, the compression collar comprising a threaded portion on its exterior surface configured to engage an interior of a second bone portion on a proximal side of the fracture site. In addition, the device includes an anti-rotational system engaging the compression collar to prevent rotation of the compression collar from its position providing the compression to the fracture site.

### TECHNICAL FIELD

The present invention relates to surgical and bone fusion devices and systems in general, and more particularly to an improved intramedullary compression device for a Jones fracture that enables the bone fusion site to be sufficiently compressed prior to fusion, and more particularly to ensure that rotation of the fusion site is prevented.

### BACKGROUND

Fractures of the foot can be problematic to treat. One reason for this may be attributed to poor blood supply to the fracture site, since bone healing relies upon good circulation, and this particular area of the bone has a notoriously poor blood supply. A fracture of the fifth metatarsal bone of the foot is sometimes referred to as a Jones fracture. The fifth metatarsal bone is at the base of the small toe, and the Jones fracture occurs in the midfoot area (the top of the bone). These fractures can either be treated with a removable cast boot or cast worn for 6 to 8 weeks, or with surgery to have a screw placed in the bone to hold the broken bone together.

A Jones fracture can be either a stress fracture (a tiny hairline break that occurs over time) or an acute (sudden) break. Jones fractures are caused by overuse, repetitive stress, or trauma. They are less common and more difficult to treat than avulsion fractures.

Other types of fractures can occur in the fifth metatarsal. Examples include avulsion fractures, mid-shaft fractures and fractures of the metatarsal head and neck. Avulsion fractures are when a small piece of bone is pulled off the main portion of the bone by a tendon or ligament. This type of fracture is typically the result of an inversion injury, in which the ankle rolls inward. Mid-shaft fractures usually result from trauma or twisting.

Surgery for a Jones fracture has about a 95% success rate and is preferable for most Jones fractures. In one treatment, a tiny puncture is made in the skin on the outside of the foot and a screw is inserted within the bone canal. The screw helps speed up the healing process. Another treatment option may be a fixator system, such as the MiniRail Fixator available from Orthofix, for maintaining compression between the bone segments externally.

In many cases, surgery may be necessary to fuse and therefore permanently immobilize the fifth metatarsal. A rod is inserted longitudinally through a hole drilled within the fifth metatarsal canal. Screws are passed laterally into the rod to hold the rod in place in the fifth metatarsal canal. When addressing Jones Fractures it is important to keep the two bone segments aligned axially as well as rotationally. While straight screws can do an adequate job of aligning bone segments axially, they typically do not do a good job of preventing rotation between the segments. Another limitation of known Jones fracture arthrodesis nailing systems is in obtaining sufficient compression across the arthrodesis site so that a proper fusion is accomplished.

Techniques in accordance with the disclosed principles provide an arthrodesis implant or intramedullary nail system and technique, wherein proper compression across the arthrodesis site can be obtained, as well as limiting the rotation between the arthrodesis segments.

### SUMMARY

An intramedullary compression device or nail is provided, as well as related methods, for a Jones fracture that enables the bone fusion site to be sufficiently compressed prior to fusion, and more particularly to ensure that rotation of the fusion site is prevented. In one embodiment, an intramedullary fixation device for a fracture is provided, and may comprise a rod having a distal section and a threaded proximal section, and means for securing the distal section within a first bone portion on a distal side of a fracture site. In addition, such a device may also comprise a compression collar threadably received over the proximal section for providing compression to the fracture site, where the compression collar comprises a threaded portion on its exterior surface configured to engage an interior of a second bone portion on a proximal side of the fracture site. Such an embodiment of a device may also include an anti-rotational system engaging the compression collar to prevent rotation of the compression collar from its position providing the compression to the fracture site.

In a more specific embodiment of an intramedullary fixation device, the device may comprise a rod having a distal section and a threaded proximal section, the rod further comprising a bend of about 10 degrees from the distal section to the proximal section from the longitudinal axis in a lateral direction. In addition, such a device may comprise at least one cross-hole in the distal section and at least one fastener adapted to be received in said cross-hole for securing the distal section within a first bone portion on a distal side of a fracture site. Furthermore, in such an embodiment, the device may include a compression collar threadably received over the proximal section for providing compression to the fracture site. The compression collar could comprise a threaded portion on its exterior surface configured to engage an interior of a second bone portion on a proximal side of the fracture site. Additionally, in such an embodiment the device may include an anti-rotational cap secured to the compression collar to prevent rotation of one cap with respect to the other cap, wherein the anti-rotational cap comprises at least one fin extending from an exterior surface and configured to engage an interior of the second bone portion to prevent rotation of the compression collar from its position providing the compression to the fracture site.

In another aspect, methods for utilizing an intramedullary fixation device to stabilize a fracture are provided. In one embodiment, such a method may comprise providing an intramedullary fixation device comprising a rod having a distal section and a threaded proximal section, and having a curvature substantially corresponding to a curvature of a human fifth metatarsal bone. In addition, such a method may include inserting the intramedullary fixation device into the intramedullary canal of the fifth metatarsal bone, and securing the distal end of the intramedullary fixation device within a first bone section on a distal side of a fracture site in the fifth metatarsal bone. Furthermore, such a method could include applying compression to the fracture by threading a compression collar over the proximal section, where the compression collar comprises a threaded portion on its exterior surface engaging an interior of a second bone portion on a proximal side of the fracture site. Additionally, such a method could include maintaining the compression of the fracture by preventing rotation of the compression collar from its position providing the compression to the fracture, where the maintaining further comprises securing the proximal end of the intramedullary fixation device to a second bone section.

### BRIEF DESCRIPTION OF THE DRAWINGS

FIG. 1 is a perspective view of a first embodiment of an intramedullary fixation device as applied to a Jones fracture in a compressed configuration.

FIG. 2 is a perspective view of the first embodiment of the intramedullary fixation device in a compressed configuration.

FIG. 3 is an exploded view of the first embodiment of the intramedullary fixation device.

FIG. 4A is a perspective view of an embodiment of a rod for use with embodiments of the intramedullary fixation device.

FIG. 4B is a top view of the rod of FIG. 4A.

FIG. 4C is a side view of the rod of FIG. 4A.

FIG. 4D is a lateral view of the rod of FIG. 4A.

FIG. 5A is a front perspective view of an embodiment of a compression collar for use with embodiments of the intramedullary fixation device.

FIG. 5B is a back perspective view of the compression collar of FIG. 5A.

FIG. 5C is a lateral view of the compression collar of FIG. 5A.

FIG. 5D is a side view of the compression collar of FIG. 5A.

FIG. 6A is a perspective view of an embodiment of a hex socket for use with embodiments of the intramedullary fixation device.

FIG. 6B is a lateral view of the hex socket of FIG. 6A.

FIG. 6C is a side view of the hex socket of FIG. 6A.

FIG. 7A is a perspective view of an embodiment of an intramedullary fixation device along with an outrigger assembly and corresponding tools.

FIG. 7B is a close-up perspective view of FIG. 7A.

FIG. 8A is a perspective view of an embodiment of the rod and the tip of an inserter in an unconnected state.

FIG. 8B is a perspective view of the rod and the tip of the inserter in a connected state.

FIG. 9A is a perspective view of the compression collar and an embodiment of the tip of an inserter in an unconnected state.

FIG. 9B is a close-up perspective view of FIG. 9A.

FIG. 10A is a perspective view of a second embodiment of the intramedullary fixation device in a compressed configuration.

FIG. 10B is a cross-sectional view from the side of the second embodiment of the intramedullary fixation device.

FIG. 11A is a top view of an embodiment of a bowed cross pin for use with embodiments of the intramedullary fixation device.

FIG. 11B is a side view of the hex socket of FIG. 11A.

FIG. 11C is a perspective view of the hex socket of FIG. 11A.

FIG. 12A is a perspective view of a third embodiment of the intramedullary fixation device in a compressed configuration.

FIG. 12B is a perspective view of a fourth embodiment of the intramedullary fixation device in a compressed configuration.

FIG. 13A is an exploded view of a fourth embodiment of the intramedullary fixation device.

FIG. 13B is a perspective view of the fourth embodiment of the intramedullary fixation device in a compressed configuration.

FIG. 13C is a cross-sectional view from the side of the fourth embodiment of the intramedullary fixation device in a compressed configuration.

FIG. 13D is a perspective view of a fourth embodiment of an intramedullary fixation device as applied to a Jones fracture in a compressed configuration.

### DETAILED DESCRIPTION

The following detailed description is of the best mode or modes of the invention presently contemplated. Such description is not intended to be understood in a limiting sense, but to be an example of the invention presented solely for illustration thereof, and by reference to which in connection with the following description and the accompanying drawings one skilled in the art may be advised of the advantages and construction of the invention. The invention is intended to cover alternatives, modifications, and equivalents, which may be included within the spirit and scope of the invention as defined by the appended claims.

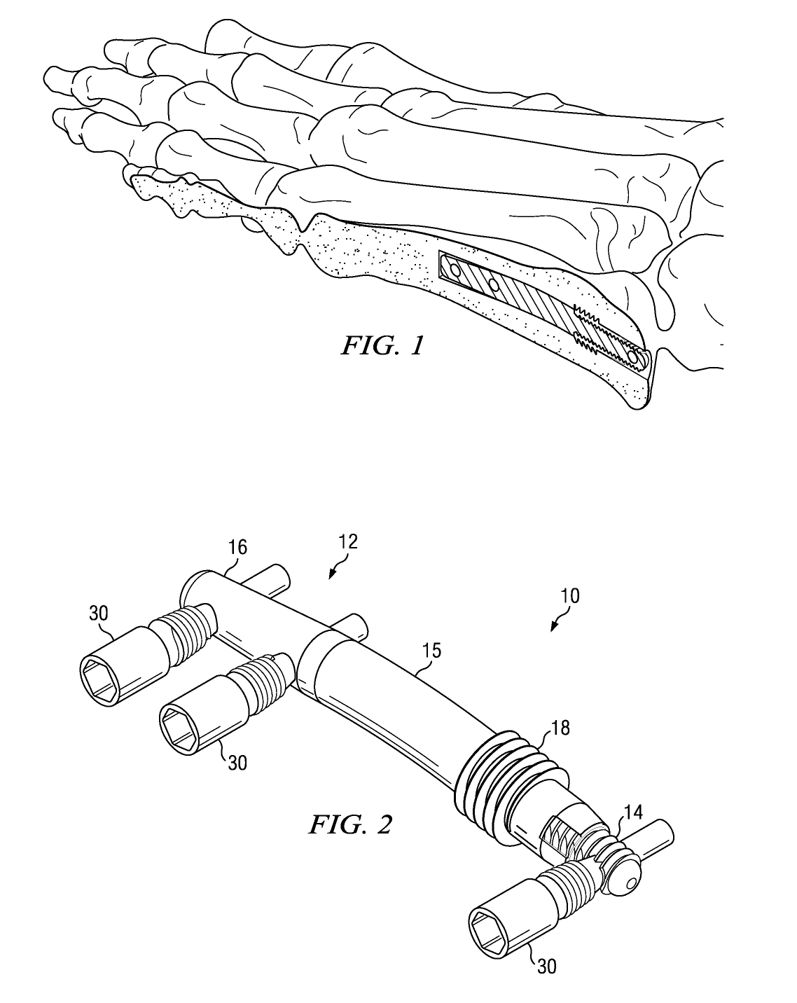

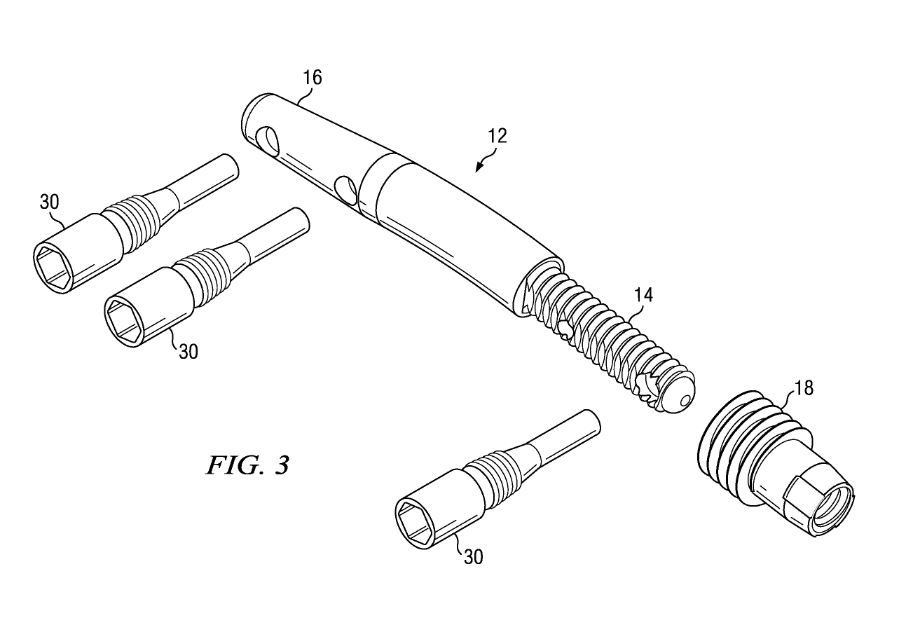

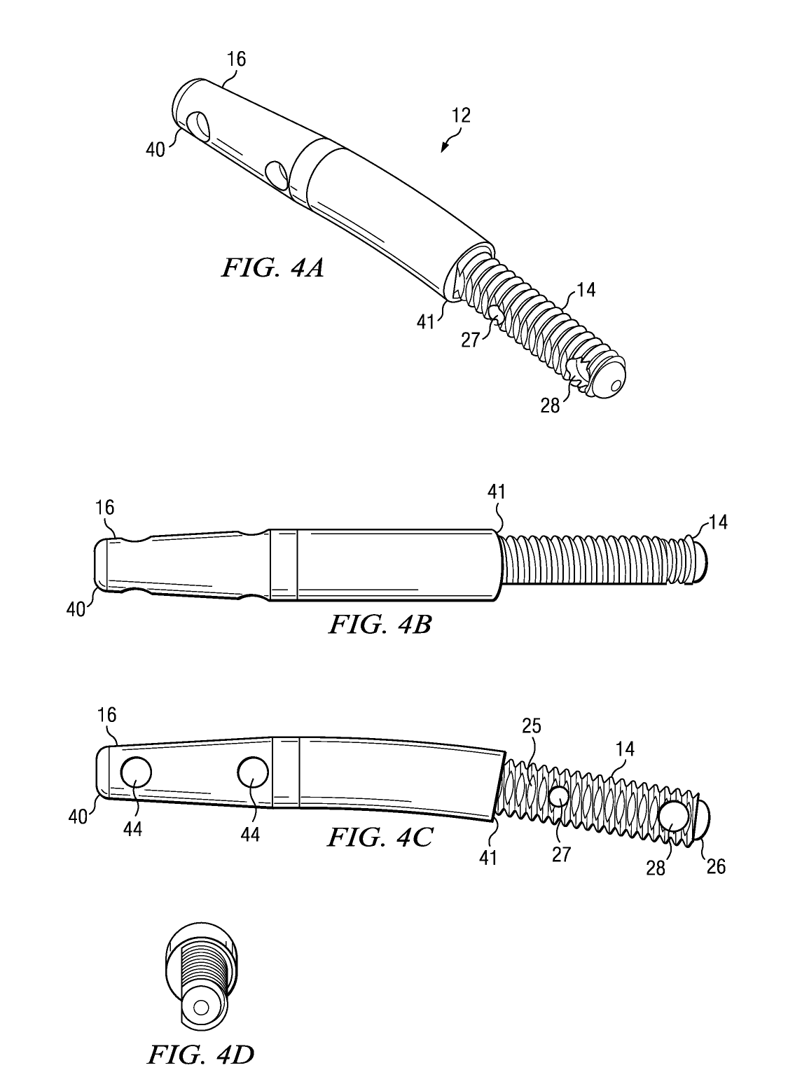

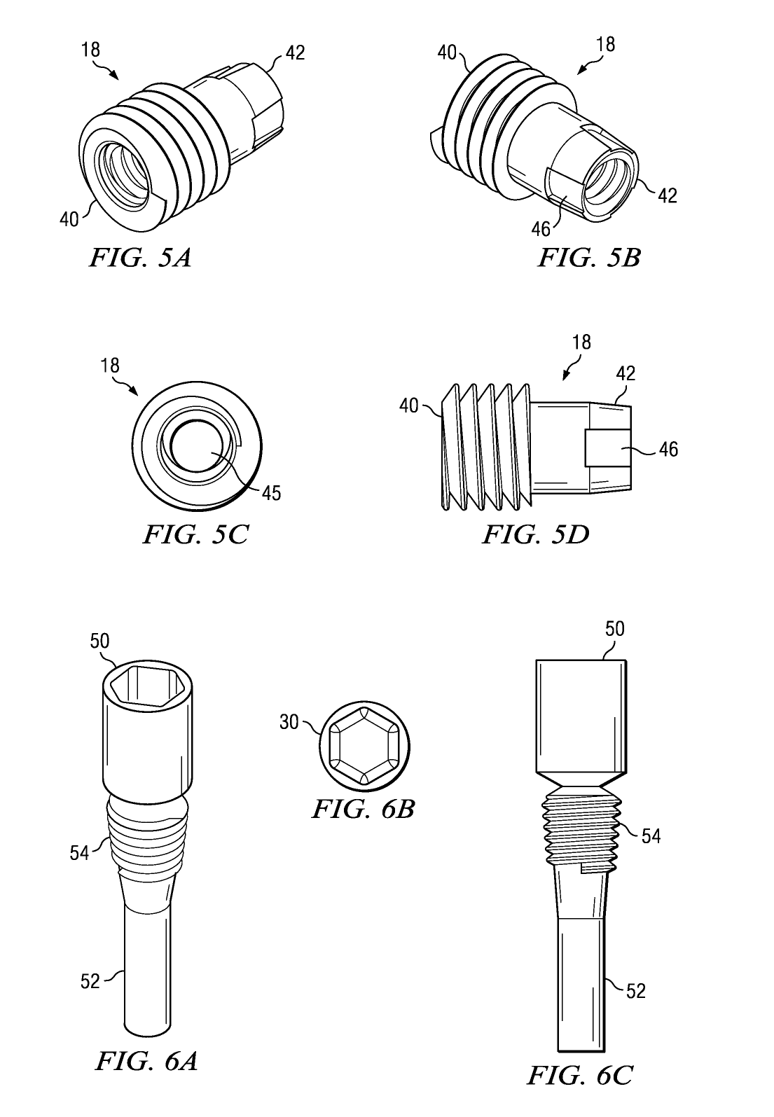

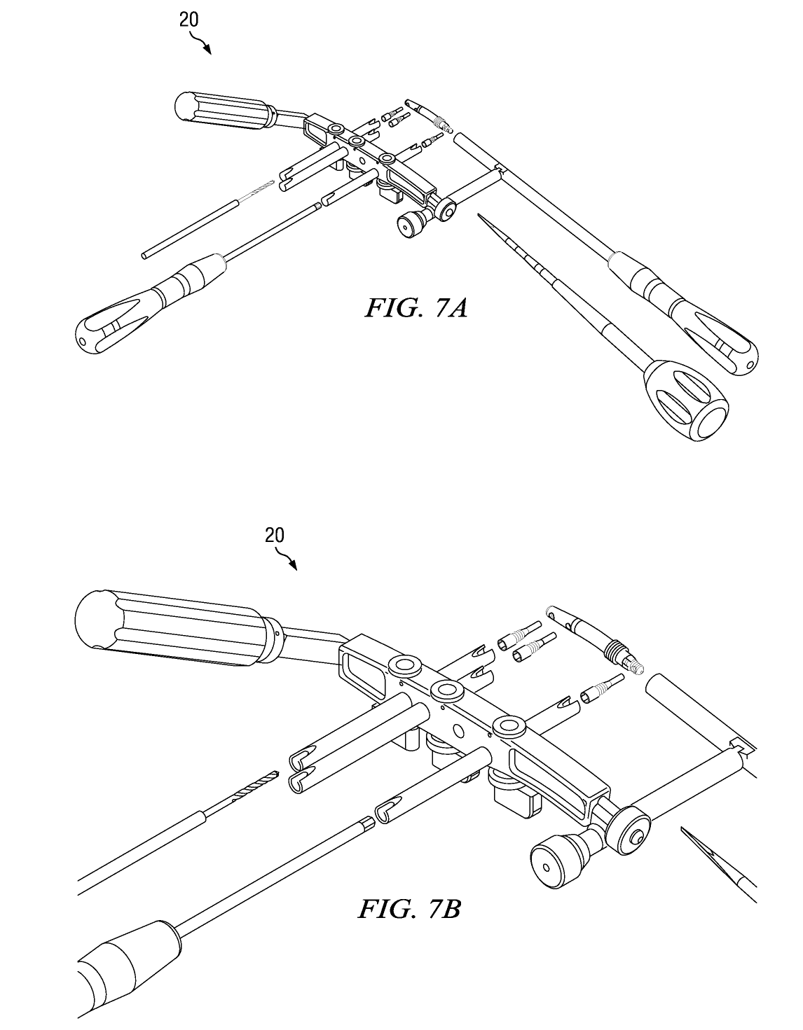

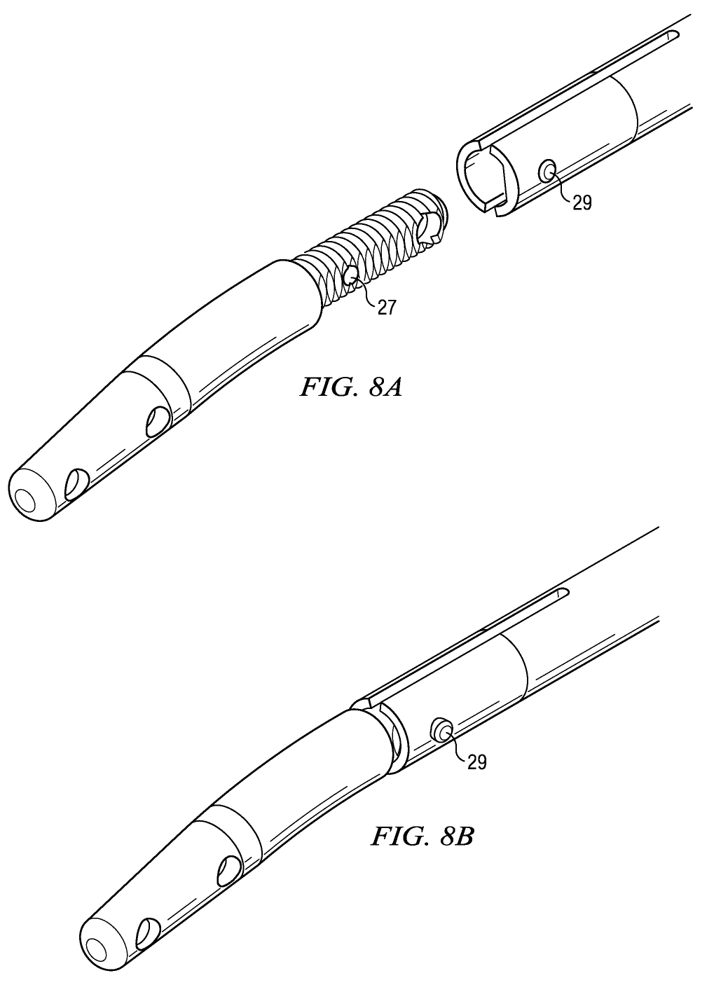

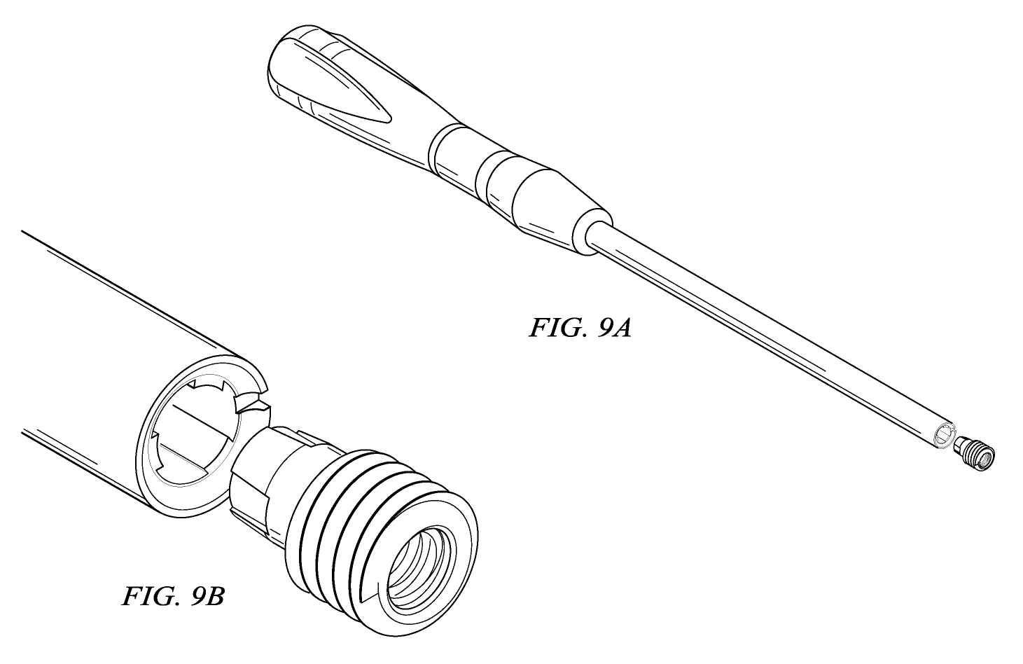

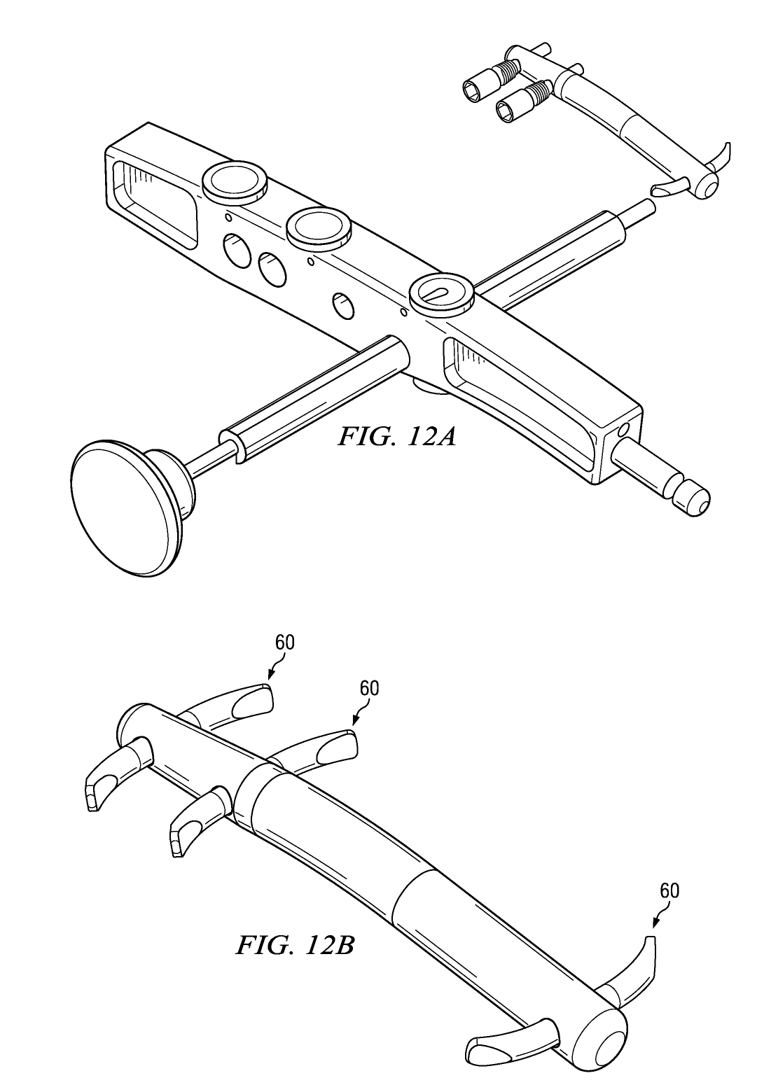

FIGS. 1-6 illustrate an embodiment of an intramedullary fixation device, FIGS. 7-9 illustrate the combination of the intramedullary fixation device and an outrigger assembly and a compression driver/inserter, and FIG. 10 illustrates a second embodiment of an intramedullary fixation device. Referring to FIG. 1, there is shown a first embodiment of an intramedullary fixation device 10 as applied to a Jones fracture in a compressed configuration. The intramedullary fixation device 10 will span the Jones fracture having a first section of bone and a second section of bone. While shown applied to a Jones fracture, the intramedullary fixation device 10 could be used at any fracture site which would benefit from the characteristics of the intramedullary fixation device 10. More particularly, the intramedullary fixation device 10 could be used anywhere to apply internal compression with anti-rotation. Although the discussion here is limited to the 5th metatarsal, in theory the intramedullary fixation device 10 could be used in any appendage, provided the access was there combined with the proper geometry. Turning to FIG. 2, intramedullary fixation device 10 includes a rod 12 (see FIGS. 4A-4D) having a proximal section 14 and a distal section 16 with an intermediate section 15 therebetween. In addition, a compression collar 18 (see FIGS. 5A-5D) is used to ensure tension or compression at the Jones fracture once the device 10 is installed, as will also be described in detail below. A plurality of screws 30 (see FIGS. 6A-6C) positions and stabilizes the intramedullary fixation device 10 to the fracture sight. The distal section 16 will be within the first section of bone and the proximal section 14 will be in the second section of bone, opposite the fracture site.

Referring more particularly now to FIGS. 4A-4D, proximal 14, intermediate 15 and distal 16 sections of rod 12 generally have a rounded or circular shape, and are preferably made of surgical stainless steel or surgical titanium, and correspond generally to the anatomical curvature of the fifth metatarsal. Of course, other advantageous material may also be employed. Moreover, although not illustrated, the rod 12 may include longitudinal flutes extending along its length. Such flutes may allow the rod 12 to pass more easily into the canal of the bone, as well as provide resistance to rotational movement of the rod 12 within the canal once in place.

In a preferred embodiment, the intermediate section 15 and the proximal section 14 as a singular component are anatomically contoured to imitate the fifth metatarsal canal. In some embodiments, the intermediate section 15 and the proximal section 14 bends from the longitudinal axis in a lateral direction for about 10 degrees. In other embodiments, the intermediate section 15 and the proximal section 14 bends from the longitudinal axis in a lateral direction ranging from about 5 to about 20 degrees.

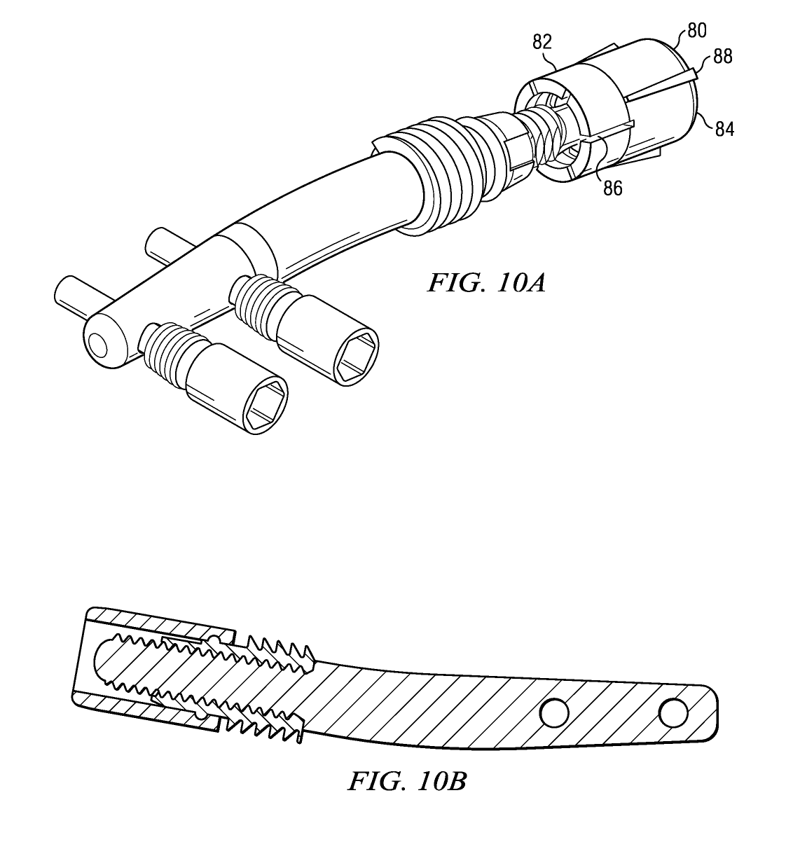

The proximal section 14 of rod 12, having a forward end 25 and a rearward end 26, has an outer surface which is preferably threaded. In some embodiments, the type of thread is cortical. In a preferred embodiment, the thread is a standard 1 mm machine pitch which will not interface with the bone. The proximal section 14 has a flat surface to allow the rod 12 to be positioned onto the compression driver 24 and be aligned with the drill fixture (see FIGS. 7A-8B). In a preferred embodiment, through-hole 28 extends perpendicularly to the longitudinal axis of the rod 12 near rearward end 26 of the proximal section 14. In a preferred embodiment, through-hole 28 supports screw 30 (see FIG. 1) which passes through proximal section 14 of rod 12 to secure the proximal section 14 to a second section of the fifth metatarsal bone. It will be understood that screws 30 may have different lengths and sizes as required and known to those skilled in the art. The rearward end 26 of proximal section 14 is sized to be used with a compression driver 24 for inserting the rod 12 into the fifth metatarsal canal. In the forward end 25 of the proximal section 14 of the rod 12 is a spherical detent 27 (sometimes referred to as a ball detent) for locating a pin 29 on the compression driver 24. The detent is used to ensure alignment and positioning of the compression driver 24 onto the rod 12. The threaded proximal section 14 preferably has a diameter ranging from about 3 mm to about 6 mm.

Distal section 16 and intermediate section 15 of rod 12 have an outer surface which may be smooth and rounded. As mentioned above, however, flutes cut into the rod 12 may extend along the distal and intermediate sections 16, 15. The distal section includes an upper end 40 which will be inserted first into the fifth metatarsal canal. The intermediate section 15 includes a lower end 41 which abuts the proximal section 14. The intermediate section 15 of rod 12 connects the distal section 16 to the proximal section 14. Through-holes 44 are situated in distal section 16 near upper end 40 extending perpendicularly to the longitudinal axis of the rod 12, and spaced apart a predetermined distance, for receiving screws 30 passed through a first section of the fifth metatarsal bone.

The distal 16 and intermediate 15 sections preferably have a diameter ranging from about 5 mm to about 6.5 mm. The overall length of the rod 12 preferably ranges from about 30 mm to about 80 mm. In some embodiments, the overall length of the rod 12 may be 44 mm or 70 mm.

Screws 30, shown in FIGS. 6A-6C, are preferably hex headed self threading screws, sized to be placed within through-holes 28 and 44 of the rod 12. The screws 30 include a top section 50 and a bottom section 52 having a threaded section 54 between them and an aperture 56 therethrough. The threaded section 54 is preferably tapered and designed to break away when sufficiently over-torqued so as to leave minimal protuberance of the top section 50 within the bone. The thread of the threaded section 54 is preferably cortical with a 0.5 mm pitch. The top section 50 has a standard hex bolt configuration for use in tightening the screw 30 into the bone to have the threaded section 54 attach to the bone to keep the rod 12 from moving. In a preferred embodiment, only the bottom section 52 will pass through the rod 12 and the threaded section 54 will have a tapered thread that engages the bone and also prevents the screw 30 from going into the rod 12. In a preferred embodiment, the top section 50 may be breakaway so as to be snapped off after insertion, leaving the threaded section 54 and bottom section 52 in the bone. In an alternate embodiment, the top section 50 is sized so that the screw 30 can be retrieved after the fracture has healed.

Referring more particularly now to FIGS. 5A-5D, compression collar 18 has a distal section 40 and a proximal section 42, and an aperture 45 therethrough along its longitudinal axis. The compression collar 18 is preferably made of surgical stainless steel or surgical titanium. Alternatively, other materials, even plastics, could be employed for the compression collar 18. Proximal section 14 of rod 12 is sized to receive compression collar 18. Accordingly, aperture 44 is sized to receive proximal section 14 of rod 12, and in preferred embodiments is a threaded engagement between these two components. Moreover, the distal section 40 is threaded both on the inside and the outside. The outer thread is a cancellous thread for cutting into the second section of bone when rotated to provide compression between the first and second section of bone. In some embodiments, the outer thread has a pitch that matches the inner thread. In other embodiments, the outer thread has a pitch that is slightly different so as to force the compression collar to bind and lock into the bone by inducing a compressive force. The inner thread may be any thread which would be complimentary to the proximal section 14 of rod 12 and would provide for the compression collar 18 to be rotated upon the proximal section 14 of rod 12. In addition, at least one alignment notch 46 is provided in proximal section 42, for receiving a corresponding tab on compression driver 24 for properly inserting and rotating the compression collar onto the rod 12, for example, in the manner described below. Although the notches 46 are shown as rectangular, they may be any shape that corresponds to the compression driver 24. The compression collar 18 preferably has a diameter ranging from about 5 mm to about 8 mm. In a preferred embodiment, the compression collar 18 has a diameter of about 6.5 mm. In addition, the compression collar 18 typically will have a length of about 7 mm when employed in a Jones fracture application.

The present system also includes an outrigger assembly 20, shown in FIGS. 7A and 7B in exploded view shown with rod 12. The outrigger assembly 20 may be a standard outrigger assembly 20 available to one skilled in the art. Alternatively, the outrigger assembly 20 may be customized for use with the disclosed nail fixation system and accompanying technique. The outrigger assembly 20 is used to properly position and align intramedullary fixation device 10 while it is being inserted and secured in the patient's fifth metatarsal canal, and then while a compressive force is applied across the fracture site. Outrigger assembly 20 is also used to properly position and align the screws 30 to secure the distal section 16 within the first section of bone. Outrigger assembly 20 is also used to properly position and align the compression driver 24 for inserting the compression collar 18 onto the proximal section 14 of rod 12. Although one compression driver 24 is shown, the compression driver 24 may have plurality of shafts for various tasks. These shafts may be interchangeable on the compression driver 24, with each having a specific function. These functions may include reaming the bone canal, inserting the rod 12 in the bone canal, and inserting and rotating the compression collar 18. It may also include installing an anti-rotation device configured to prevent rotation of the compression collar 18 once in place, as discussed in detail below.

The use of the outrigger assembly 20 and the placement of the intramedullary fixation device 10 will now be described as best shown in FIGS. 7A, 7B, 8A, 8B, 9A and 9B. While embodiments of the invention are shown for the treatment of a Jones fracture, embodiments of the invention may also be used to treat other small bone fractures.

The outrigger 20 is assembled with the rod 12, ensuring that the guide tubes align with the through-holes 28 and 44 on the rod 12. The canal of the fifth metatarsal is at least partially hollowed, which may be accomplished with an awl or similar tool. (See FIG. 7A). The compression driver 24 preferably has a tip which engages with the proximal end 14 of the rod for inserting the rod 12 into the fifth metatarsal. (See FIGS. 8A-8B). The rod 12 is inserted completely within the bone so that it transverses the fracture site, with the fracture site lying along the intermediate section 15 of the rod 12. The outrigger assembly 20 may then be mounted to the compression driver 24, which is attached to the rod 12, so that the guide tubes of the outrigger assembly 20 are properly aligned with the holes in the rod 12. (See FIG. 7B). Lateral holes are drilled to secure the distal end 16 into a first section of bone by passing screws 30 through rod 12 and into through-holes 28.

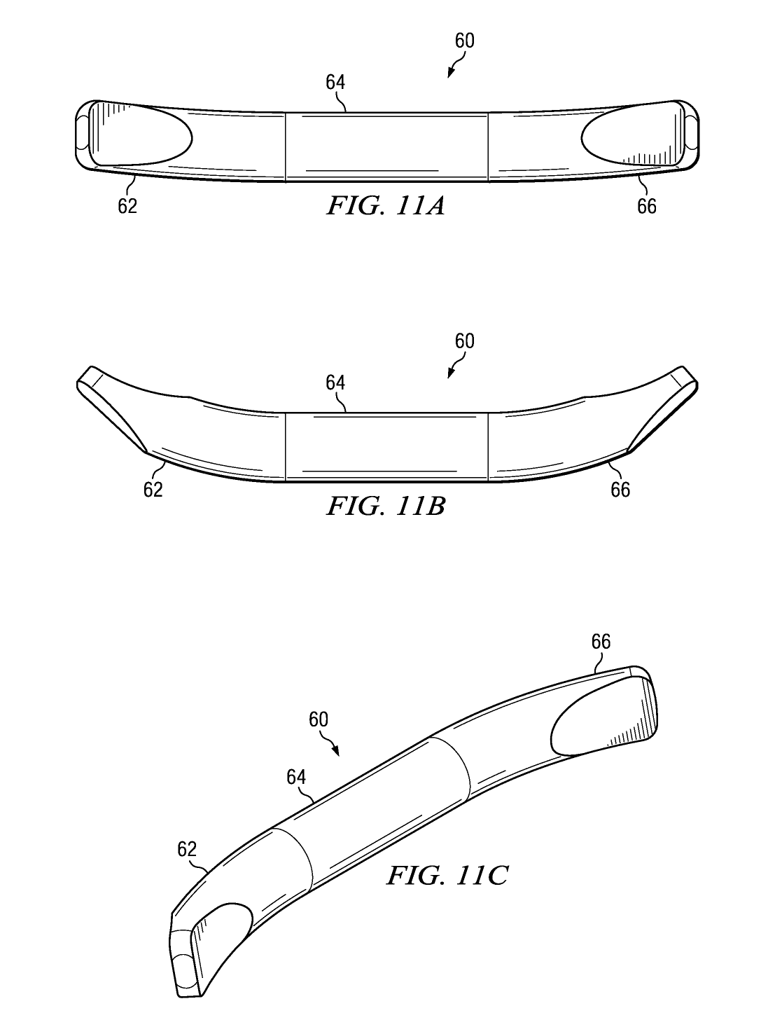

Once the distal end 16 of the rod 12 is secured to the first bone section on the distal side of the fracture, the compression collar 18 is next positioned on the compression driver 24 to be installed onto the rod 12. The notches 46 on the compression collar 18 are aligned with tabs on the interior of the tip of the compression driver 24. (See FIGS. 9A-9B). The compression collar 18 is then placed on the proximal end 14 of the rod and rotated. The purpose of compression collar 18 is to provide compression at the fracture site. The cancellous threads of the compression collar 18 become engaged in the second section of bone and the movement of compression collar 18 along the proximal section 14 towards the distal end 16 (and the towards the fracture site) will cause the space between the fracture sight to be eliminated, resulting in a desirably compressed fracture site. One or more lateral holes are drilled to secure the proximal end 14 into the second, proximal section of the bone by passing a screw 30 through rod 12 and into through-holes 28. The screw 30 through the proximal end 14 not only prevents the rod 12 from rotating with respect to the second section of the bone, but also prevents the compression collar 18 from rotating back out of the bone. Specifically, the location of one of the through-holes 28 along the proximal end 14 of the rod 12 is selected such that once the compression collar 18 is fully installed and providing the proper compression of the fracture site, the entirety of the through-hole 28 is just revealed. Thus, by placing a screw 30 through this through-hole 28, the screw abuts the proximal end of the compression collar 18. As a result, the compression collar 18 cannot rotate back out of the bone along the rod 12 once this cross screw 30 is in place.

In an alternate embodiment, to prevent rotation of the rod 12, an anti-rotation cap 80, as seen in FIGS. 10A-10B, is installed onto the proximal section 14 of the rod 12. The anti-rotation cap 80 has a front end 82 and a rear end 84, as well as an aperture therethrough. The front end 82 is sized to be engaged with the proximal section 42 of the compression collar 18. In the illustrated embodiment, tabs within the front end 82 correspond to the notches 46 of the compression collar 18. By engaging with the compression collar 18 using the tabs or other means, the anti-rotation cap 80 no longer rotates separately from compression collar 18. The front end 82 also includes one or more expansion joints 86 that flex when the anti-rotation cap 80 is placed over the proximal section 42 of the compression collar 18. For example, an O-ring or other feature may protrude from the proximal section 42 of the compression collar 18, and which is engaged in a snapped relationship by the front end 82 of the anti-rotation cap 80. Of course, other features and means for engaging the anti-rotation cap 80 to the compression collar 18 are also within the broad scope of the present disclosure. The rear end 84 of the cap includes one or more fins 88. The fins 88 secure the proximal end 14 within the bone to keep the anti-rotation cap 80 from rotating within the bone. Consequently, since the anti-rotation cap 80 is engaged with the compression collar 18, the fins 88 engaging the interior of the bone canal also prevent rotation of the compression collar 18 once installed.

To place the anti-rotation cap 80 on the installed device, the rod 12 is inserted as described above, except the lateral holes are not drilled in the proximal section 14 and the screws 30 in the proximal section 14 are not inserted. An external fastener may also provided on the proximal section 42 of the compression collar 18 to prevent the anti-rotation cap 80 from abutting the compression collar 18. In a preferred embodiment, the external fastener is the O-ring described above. The anti-rotation cap 80 may be manually forced over the proximal section 14 and onto the proximal section 42 of the compression collar 18. The notches 46 in the compression collar 18 are aligned with the tabs or other features provided in the anti-rotation cap 80. The joints 86 have the ability to flex so that the anti-rotation cap 80 can be placed in proper alignment over the compression collar 18. In an alternate embodiment, the compression driver 24 may be used to laterally insert the anti-rotation cap 80 onto the compression collar 18.

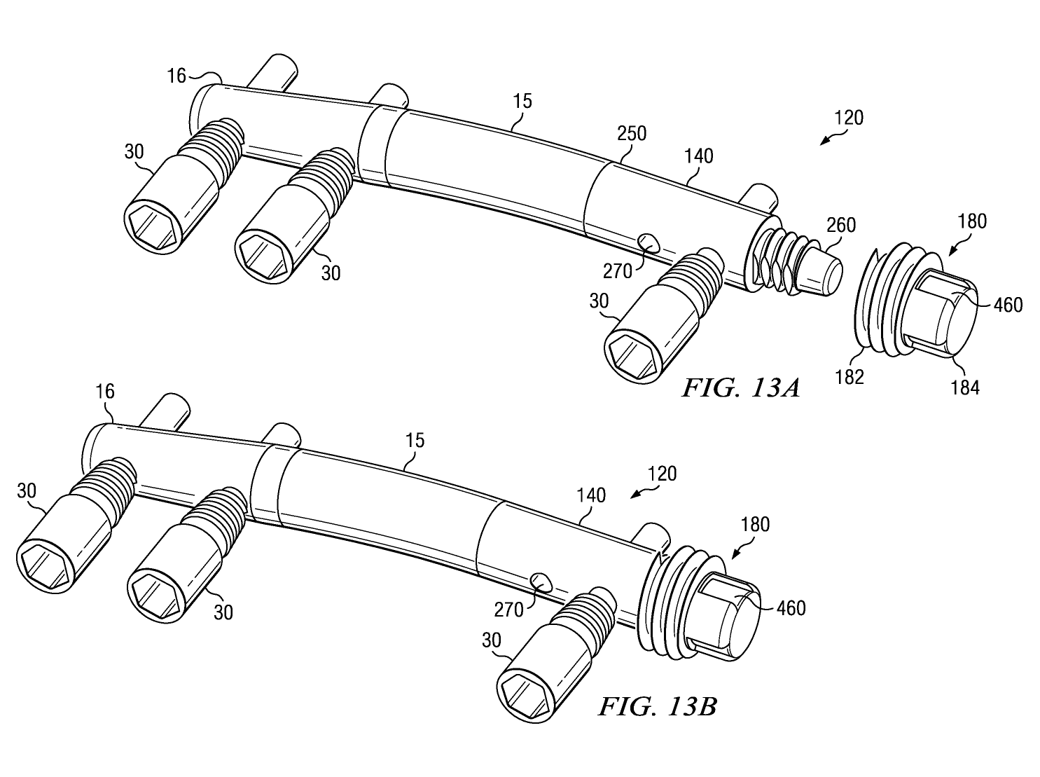

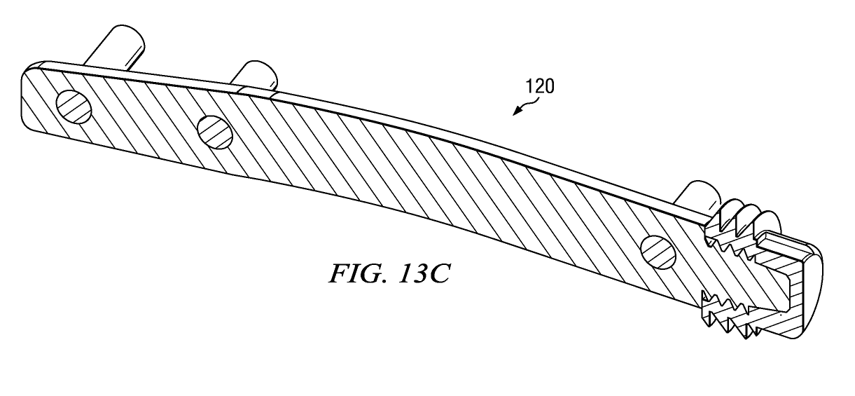

Screws 30, shown in FIGS. 11A-11C, may also be cross-pins 60, sized to be placed within through-holes 28 and 44 of the rod 12. In some embodiments, the cross-pin 60 includes a first section 62, a middle section 64, and a second section 66. The middle section 64 connects the first section 62 to the second section 66. In some embodiments, the cross-pins 60 are bowed prior to insertion and may be made of medical grade nitinol or other biocompatible shape memory alloys embodying either features of ‘memory’ or ‘superelasticity. In a preferred embodiment, the pins 60 will be custom made. In such embodiments, the bowed cross pins 60 may be inserted through a straight guide into the rod 12, as shown in FIG. 12A. After insertion, the cross-pins 60 may be allowed to bow due to their natural curvature, creating compression across the fracture site. In an alternate embodiment, the cross-pins 60 may be made of a shape memory alloy, such as, but not limited to Nitinol. Such pins may be manufactured by Memry, Inc., but again they may also be custom made or from any other origin. The pins 60 could be cooled prior to insertion into the rod 12 to straighten the pins 60 and allowed to warm up after insertion to create the bow. The cross-pins 60 may also be sized so that they can be retrieved after the fracture has healed.

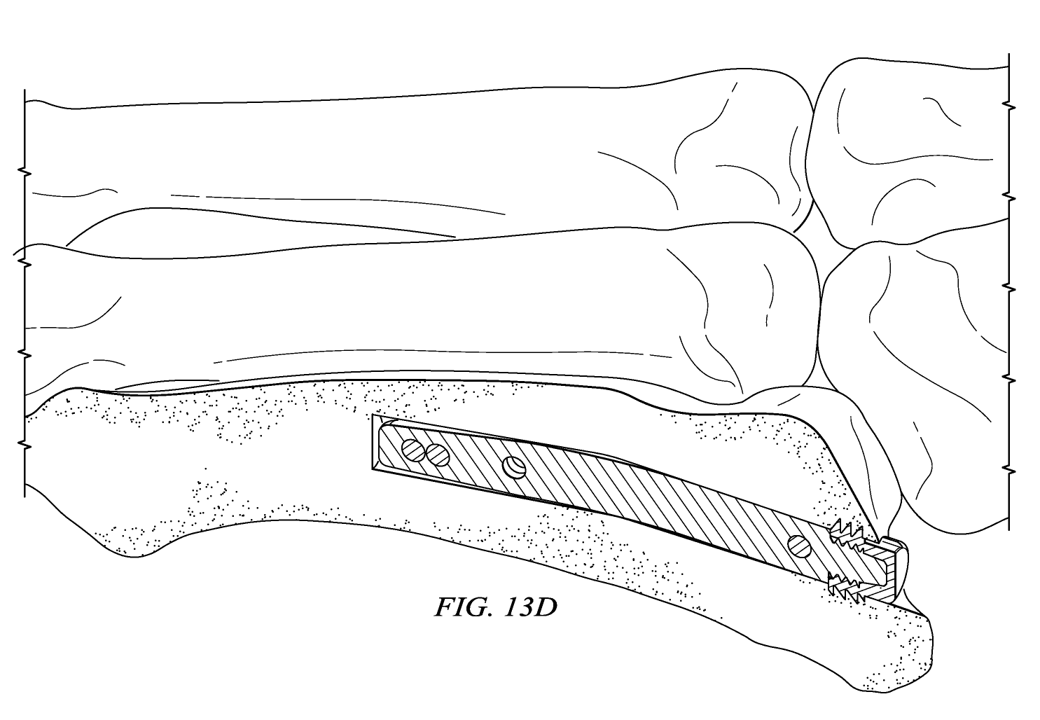

Referring to FIGS. 13A-13D, a fourth embodiment of the intramedullary fixation device 10 includes a rod 120 having a proximal section 140 and a distal section 16 with an intermediate section 15 therebetween. Components having the same reference number may be assumed to be identical as described before. Proximal 140, intermediate 15 and distal 16 sections of rod 120 generally have a rounded or circular shape, and are preferably made of surgical stainless steel or surgical titanium, and correspond generally to the anatomical curvature of a bone. In a preferred embodiment, the intermediate section 15 and the proximal section 140 as a singular component are anatomically contoured to imitate the fifth metatarsal canal. In some embodiments, the intermediate section 15 and the proximal section 140 bends from the longitudinal axis in a lateral direction for about 10 degrees. In other embodiments, the intermediate section 15 and the proximal section 140 bends from the longitudinal axis in a lateral direction ranging from about 5 to about 20 degrees. Of course, as with all embodiments constructed in accordance with the disclosed principles, the disclosed devices may also have the curvature of any other bone for which the device will be employed to stabilize a fracture.

In addition, a locking cap 180 is used to ensure tension or compression at the fracture site once the device 10 is installed, as will also be described in detail below. A plurality of screws 30 (see FIGS. 6A-6C) positions and stabilizes the intramedullary fixation device 10 to the fracture site. The distal section 16 will be within the first section of bone and the proximal section 140 will be in the second section of bone, opposite the fracture site.

The proximal section 140 of rod 120 includes a forward end 250 having a smooth outer surface and a rearward end 260 preferably having a portion with a threaded outer surface and a portion having a taper, preferably a Morse taper. In some embodiments, the type of thread is cortical. In a preferred embodiment, the thread is a standard 1 mm machine pitch which will not interface with the bone. The forward end 250 of the proximal preferably has a diameter ranging from about 5 mm to about 6.5 mm. In a preferred embodiment, through-hole 280 extends perpendicularly to the longitudinal axis of the rod 120 near the back end of the forward end 250 of the proximal section 140. In the forward end 250 of the proximal section 140 of the rod 120 is a spherical detent 270 (sometimes referred to as a ball detent) for locating a pin 29 on the compression driver 24 (See FIGS. 8A-8B). The detent is used to ensure alignment and positioning of the compression driver 24 onto the rod 120. In a preferred embodiment, through-hole 280 supports screw 30 which passes through proximal section 140 of rod 120 to secure the proximal section 140 to a second section of the fifth metatarsal bone. The rearward end 260 of proximal section 140 is sized to be used with a compression driver 24 for inserting the rod 120 into the fifth metatarsal canal. The rearward end 260 preferably has a diameter ranging from about 3 mm to about 6 mm. The overall length of the rod 120 preferably ranges from about 30 mm to about 80 mm. In some embodiments, the overall length of the rod 120 may be 44 mm or 70 mm.

After the rod 120 is inserted into the firth metatarsal canal (using a guide similar to that shown in FIGS. 7A-7B), a locking cap 180 is installed onto the proximal section 140 of the rod 120. The locking cap 180 has a front end 182 and a rear end 184, as well as an aperture therein. The aperture within the locking cap 180 is sized to be engaged with the proximal section 140 of the rod 120. The inside of the front end 182 includes threads which will mate with the threaded section of the rearward end 260 of the proximal section 140 of the rod 120. The inside of the rear end 184 is sized to mate with the taper of the proximal section 140 of the rod 120. In a preferred embodiment, the inside of the rear end 184 is a Morse taper. The outside of the rear end 184 include notches 460 for use with the compression driver when installing the locking cap 180. The mating of the threads of the threaded section of the rearward end 260 of the proximal section 140 of the rod 120 and the threads of the locking cap 180, preferably provide 3 mm of compression. The outside of the front end 182 include cancellous threads which become engaged in the second section of bone and the movement of locking cap 180 along the proximal section 140 towards the distal end 16 (and the towards the fracture site) will cause the space between the fracture sight to be eliminated, resulting in a desirably compressed fracture site.

While various embodiments of the disclosed principles have been described above, it should be understood that they have been presented by way of example only, and not limitation. Thus, the breadth and scope of the invention(s) should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically and by way of example, although the headings refer to a “Technical Field,” such claims should not be limited by the language chosen under this heading to describe the so-called technical field. Further, a description of a technology in the “Background” is not to be construed as an admission that technology is prior art to any invention(s) in this disclosure. Neither is the “Summary” to be considered as a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

We claim:

1. An intramedullary fixation device for a fracture, the device comprising:
(a) a rod having a distal section and a threaded proximal section;
(b) means for securing the distal section within a first bone portion on a distal side of a fracture site;
(c) a compression collar threadably received over the proximal section for providing compression to the fracture site, the compression collar comprising a threaded portion on its exterior surface configured to engage an interior of a second bone portion on a proximal side of the fracture site; and
(d) an anti-rotational system engaging the compression collar to prevent rotation of the compression collar from its position providing the compression to the fracture site.

2. The device of claim 1, wherein the rod further comprises a bend of about 10 degrees from the distal section to the proximal section from the longitudinal axis in a lateral direction.

3. The device of claim 1, wherein the compression collar further includes external features adapted to receive a driving device to facilitate rotating the compression collar about the proximal section.

4. The device of claim 1, wherein the means for securing the distal section comprises at least one cross-hole in the distal section and at least one screw adapted to be received in said cross-hole.

5. The device of claim 1, wherein the means for securing the distal section comprises at least one cross-hole in the distal section and at least one bowed cross-pin adapted to be received in said cross-hole.

6. The device of claim 1, wherein the anti-rotational system comprises at least one cross-hole through the proximal section and at least one screw adapted to be received in said hole and abutting a proximal face of the compression collar.

7. The device of claim 1, wherein the anti-rotational system comprises at least one cross-hole through the proximal section and at least one bowed cross-pin adapted to be received in said hole and abutting a proximal face of the compression collar.

8. The device of claim 1, wherein the anti-rotational system comprises an anti-rotational cap adapted to be secured to the compression collar to prevent rotation of one cap with respect to the other cap.

9. The device of claim 8, wherein the compression collar further comprises at least one notch and the anti-rotational cap comprises at least one corresponding tab for aligning the anti-rotational cap with the compression collar.

10. The device of claim 8, wherein the anti-rotational cap further comprises at least one fin extending from an exterior surface and configured to engage an interior of the second bone portion.

11. The device of claim 8, wherein the anti-rotational cap further comprises flexible tabs configured to engage a corresponding external feature on the compression collar in a snapping relationship.

12. The device of claim 8, wherein the anti-rotational cap further includes features adapted to receive a driving device to facilitate securement of the anti-rotational cap to the compression collar.

13. The device of claim 1, further comprising an outrigger assembly attachable to the rod, the outrigger assembly for positioning the means for securing the distal section within the first bone portion, the compression collar, and the anti-rotation system of the device.

14. The device of claim 1, wherein the threaded portion on the exterior surface of the compression collar comprises a cancellous threaded portion.

15. The device of claim 1, wherein a pitch of the threaded portion on the exterior surface of the compression collar is substantially equal to a pitch of the threaded portion of the proximal section.

16. An intramedullary fixation device for a fracture, the device comprising:
(a) a rod having a distal section and a threaded proximal section, the rod further comprising a bend of about 10 degrees from the distal section to the proximal section from the longitudinal axis in a lateral direction;
(b) at least one cross-hole in the distal section and at least one fastener adapted to be received in said cross-hole for securing the distal section within a first bone portion on a distal side of a fracture site;
(c) a compression collar threadably received over the proximal section for providing compression to the fracture site, the compression collar comprising a threaded portion on its exterior surface configured to engage an interior of a second bone portion on a proximal side of the fracture site; and
(d) an anti-rotational cap secured to the compression collar to prevent rotation of one cap with respect to the other cap, wherein the anti-rotational cap comprises at least one fin extending from an exterior surface and configured to engage an interior of the second bone portion to prevent rotation of the compression collar from its position providing the compression to the fracture site.

17. The device of claim 16, wherein the compression collar further includes external features adapted to receive a driving device to facilitate rotating the compression collar about the proximal section.

18. The device of claim 16, wherein the compression collar further comprises at least one notch and the anti-rotational cap comprises at least one corresponding tab for aligning the anti-rotational cap with the compression collar.

19. The device of claim 16, wherein the anti-rotational cap further comprises flexible tabs configured to engage a corresponding external feature on the compression collar in a snapping relationship.

20. The device of claim 16, wherein the anti-rotational cap further includes features adapted to receive a driving device to facilitate securement of the anti-rotational cap to the compression collar.

21. The device of claim 16, wherein the threaded portion on the exterior surface of the compression collar comprises a cancellous threaded portion.

22. The device of claim 16, wherein a pitch of the threaded portion on the exterior surface of the compression collar is substantially equal to a pitch of the threaded portion of the proximal section.

23. The device of claim 16, wherein the fastener comprises a screw or a bowed-cross pin.

24. A method of utilizing an intramedullary fixation device to stabilize a fracture, the method comprising:
(a) providing an intramedullary fixation device comprising a rod having a distal section and a threaded proximal section, and having a curvature substantially corresponding to a curvature of a human fifth metatarsal bone;
(b) inserting the intramedullary fixation device into the intramedullary canal of the fifth metatarsal bone;
(c) securing the distal end of the intramedullary fixation device within a first bone section on a distal side of a fracture site in the fifth metatarsal bone;
(d) applying compression to the fracture by threading a compression collar over the proximal section, the compression collar comprising a threaded portion on its exterior surface engaging an interior of a second bone portion on a proximal side of the fracture site;
(e) maintaining the compression of the fracture by preventing rotation of the compression collar from its position providing the compression to the fracture, the maintaining further securing the proximal end of the intramedullary fixation device to a second bone section.

25. The method of claim 24, wherein securing the distal end of the intramedullary fixation device within a first bone section comprises placing at least one screw through a corresponding at least one cross-hole through a distal end of the device, wherein threads of the at least one screw threadedly engage the first bone portion.

26. The method of claim 24, wherein securing the distal end of the intramedullary fixation device within a first bone section comprises placing at least one bowed cross-pin through a corresponding at least one cross-hole through a distal end of the device, wherein bowed ends of the cross-pin engage the first bone portion.

27. The method of claim 24, wherein maintaining the compression of the fracture by preventing rotation of the compression collar from its position providing the compression to the fracture comprises securing an anti-rotational cap to the compression collar to prevent rotation of one cap with respect to the other cap.

28. The method of claim 27, wherein securing an anti-rotational cap to the compression collar further comprises engaging at least one notch on the compression collar with at least one corresponding tab on the anti-rotational cap to align the anti-rotational cap with the compression collar.

29. The method of claim 27, wherein securing the proximal end of the intramedullary fixation device to a second bone section comprises providing at least one fin extending from an exterior surface of the anti-rotational cap, the at least one fin engaging an interior of the second bone portion.

30. The method of claim 24, wherein maintaining the compression of the fracture by preventing rotation of the compression collar from its position providing the compression to the fracture comprises placing a screw within a cross-hole through the proximal section such that a portion of the screw abuts a proximal face of the compression collar

31. The method of claim 30, wherein securing the proximal end of the intramedullary fixation device to the second bone section comprises the screw threadedly engaging the second bone portion.

32. The method of claim 24, wherein maintaining the compression of the fracture by preventing rotation of the compression collar from its position providing the compression to the fracture comprises placing a cross-pin within a cross-hole through the proximal section, and allowing the cross-pin to reached a final, bow shape such that a central portion of the bowed cross-pin bows against a proximal face of the compression collar while ends of the bowed cross-pin engage the second bone portion.

33. An intramedullary fixation device for a fracture, the device comprising:
(a) a rod having a distal section and a threaded proximal section;
(b) means for securing the distal section within a first bone portion on a distal side of a fracture site;
(c) means for securing the proximal section within a second bone portion on a proximal side of a fracture site; and
(d) a locking cap threadably received over the proximal section for providing compression to the fracture site and to prevent rotation of the rod from its position, the locking cap comprising a threaded portion on its exterior surface configured to engage an interior of a second bone portion on a proximal side of the fracture site.
